(19) United States
(12) Patent Application Publication
(10) Pub. No.: US 2004/0116907 A1
(43) Pub. Date: Jun. 17, 2004

(54) Surgical marker

(76) Inventor: Arthur A. Tartaglia, Richmond Heights, OH (US)

Correspondence Address: Scott A. McCollister, Fay, Sharpe, Fagan, Minnich & McKee, LLP, Seventh Floor, 1100 Superior Avenue, Cleveland, OH, 44114-2579, US

(21) Appl. No.: 10/657,889

(22) Filed: Sep. 9, 2003

Related U.S. Application Data
(60) Provisional application No. 60/409,395, filed on Sep. 10, 2002.

(51) Int. Cl. A61B 17/00

(52) U.S. Cl. 606/1; 606/116

(57) ABSTRACT

A marking pen for indicating the proper location of incisions to be made on a patient undergoing surgery is provided. The marking pen includes a handle having a first end and a second end; a wheel comprising a gelatinous ink-soaked polymer rotably attached to the first end of the handle; and a plurality of treads on a periphery of the wheel.

This application claims priority under 35 U.S.C. § 119(e) to U.S. Provisional Application Serial No. 60/409,395 filed Sep. 10, 2002, which is incorporated by reference.

### BACKGROUND OF THE INVENTION

The present invention relates, in general, to a marking pen. More particularly, to a marking pen for use during surgery, so as to guide the surgeon in making a proper incision. The pen may also aid the surgeon in marking where to place sutures, staples, glue or other mechanisms for closing an incision post operatively.

The use of marking pens in surgery is common. A surgeon will mark lines on a patient's body so as to know the proper place and length of the incision that will be made during the operation. Such lines can be particularly important in plastic surgery. In such cases, it is extremely important that the incisions be at the proper location, and of the appropriate length. This is often difficult to do during surgery without the aid of markings.

Typically, a surgeon will use a felt tip pen to mark lines representing the desired incisions. Felt tip pens can drag across the patient's skin catching some of the skin. The skin that is caught by the pen can bunch up underneath the pen resulting in a jagged or crooked line. Also, felt tip pens, in particular, can “bleed” when they are first placed on a patient's skin.

Marking pens of the prior art also have a tendency to dry out. Prior art pens have tried to solve this problem of drying out by providing a surgical marker with a reservoir that feeds a marking portion of the surgical marker. In these pens having reservoirs, the channels leading to the marking portion can become clogged.

Due to these problems with marking pens, some surgeons rely on other marking techniques. Some doctors dip toothpicks or other similar instruments in ink, and then drag the ink-soaked toothpick across the patient's skin to form a line. The toothpicks, or other similar marking instruments do not hold a significant amount of ink and must be repeatedly dipped into the ink when the surgeon is drawing a number of lines.

Accordingly, to overcome these problems, there is a need for a marking pen that enables a surgeon to make a line on the patient's skin that is not jagged. Additionally, the line can provide configurations that can mark a patient's skin to aid the doctor in the placement of sutures after the surgery has been performed.

### SUMMARY OF THE INVENTION

In accordance with one aspect of the invention, a marking pen for indicating the proper location of incisions to be made on a patient undergoing surgery is provided. The marking pen includes a handle having a first end and a second end; a wheel comprising a gelatinous ink-soaked polymer rotably attached to the first end of the handle; and a plurality of treads on a periphery of the wheel.

In accordance with another aspect of the invention, a method of marking surgical lines on the skin of a human is provided. The method comprises providing a wheel having cross-shaped treads; marking the skin with the wheel forming a first line aligned with the circumference of the wheel; and marking the skin with the wheel forming a plurality of hash-marks substantially perpendicular to the first line.

The advantages and benefits of the present invention will become apparent to those of ordinary skill in the art upon reading and understanding the following detailed description of the preferred embodiments.

### BRIEF DESCRIPTION OF THE DRAWINGS

The drawings are only for purposes of illustrating preferred embodiments, and are not to be construed as limiting the invention. The invention may take form in various components and arrangement of components, and in various steps and arrangement of steps, preferred embodiments of which will be illustrated in the accompanying drawings.

FIG. 1 is a perspective view of a first embodiment of the surgical marker in accordance with the invention.

FIG. 2 is a perspective view of a first end of the surgical marker shown in FIG. 1 with a marking wheel removed.

FIG. 3 is a perspective view of the first end of the surgical marker of FIG. 1 showing a first embodiment of the marking wheel.

FIG. 4 is a perspective view of the first end of the surgical marker of FIG. 1 showing a second embodiment of the marking wheel.

FIG. 5 is a perspective view of a second embodiment of the surgical marker in accordance which the invention.

FIG. 6 is a perspective view of a first end of the surgical marker of FIG. 5.

### DETAILED DESCRIPTION OF THE INVENTION

It is to be understood that the specific devices and processes illustrated in the attached drawings, and described in the following specification are simply exemplary embodiments of the inventive concepts. Therefore, specific examples and characteristics relating to the embodiments disclosed herein are not to be considered as limiting.

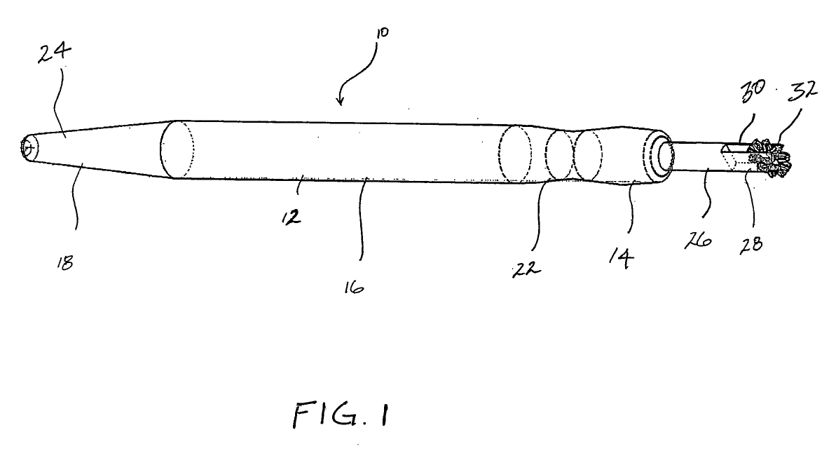

Referring to FIG. 1, a surgical marker shown generally at 10 includes a body 12 having a first end portion 14 a central portion 16 and a second end portion 18. The body 12 can make up at least a portion of a handle of the surgical marker. The body has a generally cylindrical shape and can be made of any conventional material, preferably plastic. Since this surgical marker does not require a reservoir, the body can be solid. First end portion 14 is cylindrically shaped as is the central portion 16. A contoured portion 22 is located at the conjunction of the central portion and the first end portion. The contoured portion 22 can provide a more ergonomic handle for the surgical marker. The second end portion 18 has a tapered end 24 that tapers away from the central portion 16.

A neck 26 attaches to the first end portion 14 of the body. The neck can either be attached by fastening or it can be an integral unit with the body 12. The neck is cylindrical having two arms, 28 and 30, that project away from the first end portion 14. A wheel 32 is rotably attached between the arms 28, 30. The neck and the arms can also be made from a solid piece of plastic, containing no channels inside of them. The neck can be detachable from the body so that the neck and wheel can be discarded and the remainder of the marker can be re-used.

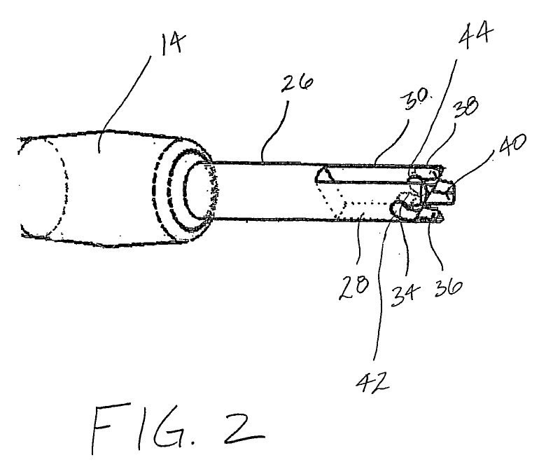

Referring now to FIG. 2, arm 28 includes two forks 34 and 36 and arm 30 includes two forks 38 and 40. Forks 34 and 36 and arm 28 define an arcuate notch 42 that can receive an axle of wheel 32 (FIG. 1). Arm 30 and forks 38 and 40 define an arcuate notch 44. Arcuate notch also receives an axle of the wheel 32 shown in FIGS. 1, 3 and 4. The arcuate portions 42, 44 are adapted to surround more than half the periphery of the axle of the wheel. Thus, the wheel can lock into the notch when mounted to the neck, yet the wheel can also be detached from the neck after use.

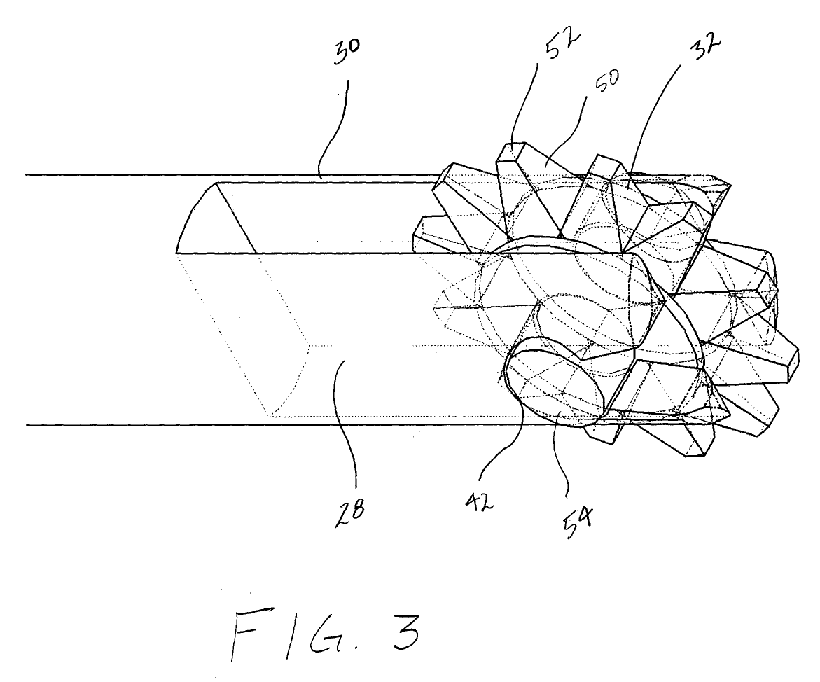

Referring now to FIG. 3, the wheel 32 includes a plurality of treads 50 located on a peripheral surface of the wheel. The wheel can be made of a conventional material including an elastomer, a plastic, or other well known material. The wheel in the preferred embodiment is made of a gelatinous ink-soaked polymer. More specifically the wheel comprises a plasticized (approximately 60% ink and approximately 40% resin) felt tip from the medical industry. Such polymers are available from Identity Group, 1480 Gould Drive, Cookeville, Tenn. and the Bacon Felt Co. Other ink-soaked resins could also be used including, but not limited to, polyethylene and polypropylene. The ink-soaked polymer is preferred because it reduces the tendency of the wheel to drag across the patient's skin, catching some of the skin and causing the skin to “bunch” together. Furthermore, the ink-soaked polymer wheel does not have a tendency to dry out. Also, the ink-soaked polymer wheel eliminates the need for a ink reservoir where channels leading from the reservoir to the wheel may clog. Also, a surgeon using such an ink-soaked polymeric wheel, when marking lines on the patient's skin, does not need to dip the surgical marker in ink prior to marking the patient's skin.

The marking agent or ink used with the wheel can be any conventional marking agent used to mark a patient's skin. In the present invention the preferred marking agent is Gentian Violet, however other marking agents can be used.

The wheel 32 includes treads 50 having marking surfaces 52 disposed at a peripheral edge of the treads. The treads 50 are the shape of a frustum of a prism, however, the treads could take form in a number of different shapes. The marking surfaces 52 are spaced from one another resulting in a dotted line on the patient's skin when the surgeon makes the mark, however, the marking surfaces need not be spaced from one another when a continuous line is desired. The marking surfaces can be flat, or arcuate to match the radius of the wheel so that as large amount of marking surface as possible contacts the patient's skin. The marking surface can be square, circular or any other shape. The dots that are made by the wheel on the patient's skin can be spaced apart from one another the conventional distance at which post-operative sutures are placed, or some multiple thereof, i.e. every third dot a suture or other device to close the incision is placed.

An axle 54 is disposed in the center of the wheel 32. The axle 54 can be separate from the wheel, or the wheel and axle can be made from one integral unit. As stated before, the axle is received in the arcuate notches 42, 44 of the arms 28, 30. The receipt of the axle in the notches allows the wheel to detach from the neck so that the remaining portion of the surgical marker can be re-used.

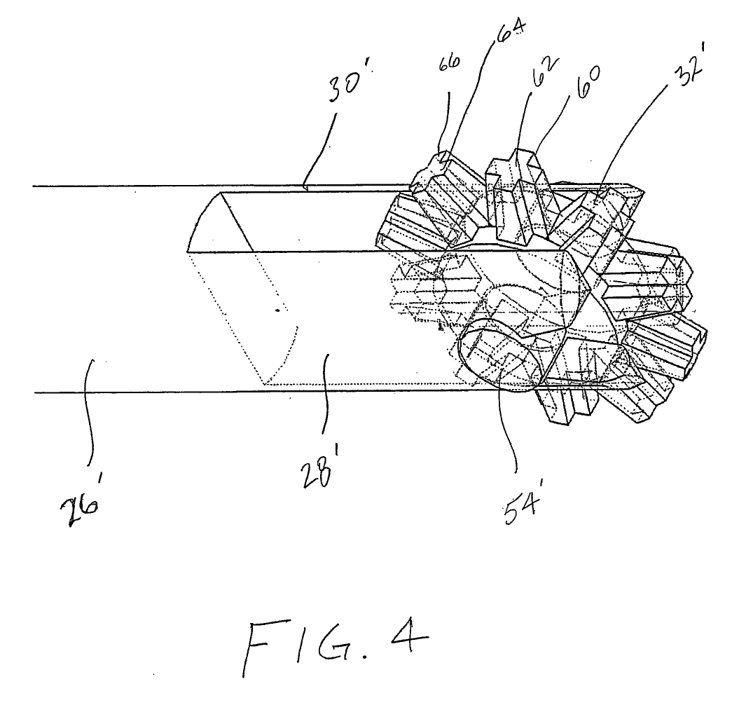

With reference to FIG. 4, another embodiment of a wheel of a surgical marker is shown. For ease of illustration and comprehension of this alternative, like components are identified with like numerals with a primed suffix (′) and new components are identified by new numerals.

A wheel 32′ is rotably mounted to a neck 26′ between arms 28′ and 30′. An axle 54′ of the wheel is received in the arms 28′ and 30′. The wheel includes a plurality of treads 60 having markings surfaces 62. The marking surfaces 62 are cross-shaped having a first portion 64 aligned with a circumference of the wheel and a second portion 66 aligned perpendicular to the circumference of the wheel. The marking surfaces 62 are spaced from one another, similar to the marking surfaces 52 of FIG. 3. The marking surface can be flat, rounded to match the radius of the wheel, or other configuration.

When the surgeon draws the line prior to incision, the first portion 64 of the marking surfaces creates a dotted line to guide the surgeon when making the incision. The second portion 66 of the marking surface creates a hash mark substantially perpendicular to the line formed by the first portion. The hash marks created by the second portion 66 provide a guide to the surgeon when closing the incision. Accordingly, the second portions 66 are spaced apart from one another such that when a line is drawn on patient's skin, hash marks are formed a distance from one another that is conventional for the spacing of sutures, or some multiple thereof. The radius of the wheel 32′ as measured to the marking surface of the tread and the angle measured between two adjacent treads is controlled by the distance at which the doctor will place sutures post-operatively and vice-versa.

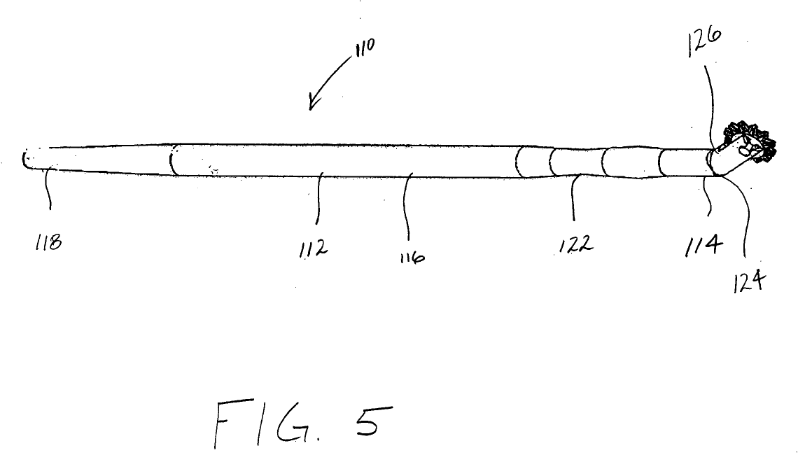

Referring now to FIG. 5, a surgical marker is generally shown at 110. The surgical marker includes a body 112 having a first end 114, a central portion 116 and a second end 118. The body 112 or handle also includes a contoured portion 122 located at the conjunction of the first end 114 and the central portion 116. The body or handle portion of the surgical marker 110 is very similar in construction to the marker 10 described above. The surgical marker 112 also includes a bend 124 where a neck 126 joins the body at the first end 114. The bend shown in FIG. 5 is a 30° degree bend; however, other degrees of bend are contemplated by the scope of the invention.

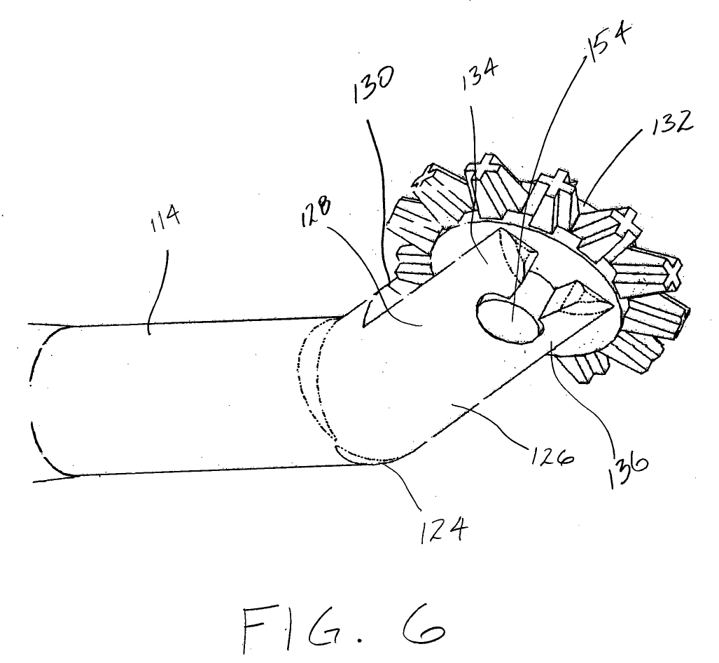

Referring to FIG. 6, the neck 126 includes a first arm 128 and a second arm 130. The first arm 128 is spaced from the second arm 130 so that the arms can receive a wheel 132. The wheel 132 is similar to the wheel previously described in FIGS. 1, 3 and 4. The wheel is rotably mounted to the arms 128 and 130 by an axle 154. The wheel is received in notches defined by forks of which fork 134 and fork 136 are visible in FIG. 6.

The bend 124 of the surgical marker 110 allows the surgeon to hold more ergonomically the surgical marker when drawing lines on the patient's skin. The bend also allows the doctor to grip the marker at an angle one typically holds a pen while writing, yet the neck portion is situated substantially normal to the patient's skin. This orientation of the wheel can lessen the likelihood of the wheel catching any of the patient's skin, which can cause unwanted bunching of the skin.

The invention has been described with reference to the preferred embodiments. Obviously, modifications and alterations will occur to others upon reading and understanding the preceding detailed description. It is intended that the invention be construed as including all such modifications and alterations.

What is claimed is:

1. A marking pen for indicating the proper location of an incision to be made on a patient undergoing surgery, the pen comprising:
a handle having a first end and a second end;
a wheel rotably attached to the first end of said handle, wherein said wheel includes a plurality of treads disposed about a periphery of said wheel.
